(19) United States
(12) Patent Application Publication
(10) Pub. No.: US 2025/0215062 A1
(43) Pub. Date: Jul. 3, 2025

(54) POLYPEPTIDE ANTIGEN OF BOVINE AND SHEEP PREGNANCY-ASSOCIATED GLYCOPROTEIN AND APPLICATION THEREOF

(71) Applicants: WUHAN ELABSCIENCE BIOTECHNOLOGY CO., LTD, WUHAN (CN); WUHAN UNI-SCIENCE BIOTECHNOLOGY CO., LTD, WUHAN (CN)

(72) Inventors: YIBIN LENG, WUHAN (CN); QINQIN HU, WUHAN (CN); HONGXING XIA, WUHAN (CN); LIANG FENG, WUHAN (CN); XIANG SHU, WUHAN (CN); PING LAN, WUHAN (CN); TINGTING ZOU, WUHAN (CN)

(21) Appl. No.: 19/000,524

(22) Filed: Dec. 23, 2024

(30) Foreign Application Priority Data
Dec. 31, 2023 (CN) 202311860937.1

(51) Int. Cl. C07K 14/705

(52) U.S. Cl. CPC C07K 14/70503

(57) ABSTRACT

The present disclosure provides a polypeptide antigen of bovine and sheep pregnancy-associated glycoprotein and application thereof. The polypeptide antigen of the present disclosure, comprising two co-antigen polypeptides. Antibodies prepared by using the polypeptide antigen provided by the present disclosure can be used for simultaneously detecting early pregnancy of bovine and sheep; the test strip of the present disclosure fills the blank in the prior art, and can combine two commercial tests in a single test strip, and improves the convenience of customer use; on the other hand, the production cost of manufacturers is also reduced.

### CROSS REFERENCE OF RELATED APPLICATIONS

This patent application claims the benefit and priority of Chinese Patent Application No. 202311860937.1 filed on Dec. 31, 2023, the disclosure of which is incorporated by reference herein in its entirety as part of the present application.

### SEQUENCE LISTING

The instant application contains a Sequence Listing which has been submitted electronically in ST.26 format and is hereby incorporated by reference in its entirety. Said ST.26 copy, created on Dec. 18, 2024, is named Sequence Listing.xml and is 3,067 bytes in size.

### TECHNICAL FIELD

The present disclosure relates to the technical field of antibodies, specifically to a polypeptide antigen of bovine and sheep pregnancy-associated glycoprotein and application thereof.

### BACKGROUND

Early pregnancy-associated glycoprotein (PAG) is a commonly used diagnostic indicator in bovine and sheep for early pregnancy. By measuring the concentration changes of PAG in the blood of livestock, the pregnancy status of bovine and sheep can be judged as early as possible. The prior art provides a monoclonal antibody for pregnancy-associated glycoprotein and its application in early pregnancy detection in bovine (CN 114134123A) Still, this technology can only detect bovine as a species, which is relatively single. The same is true for other prior art reports, which are used to detect pregnancy in a single species of bovine or sheep. According to research, there are currently no reports on colloidal gold test strips for early pregnancy joint testing of bovine and sheep on the market.

### SUMMARY

To fill the gaps in existing technology, the present disclosure provides a polypeptide antigen of bovine and sheep pregnancy-associated glycoprotein and application thereof.

Specifically, the polypeptide antigen of the bovine and sheep pregnancy-associated glycoprotein of the present disclosure, comprising two co-antigen polypeptides, one of the co-antigen polypeptides comprises the amino acid sequence shown in SEQ ID NO. 1, the other comprises the amino acid sequence shown in SEQ ID NO. 2.

### The Amino Acid Sequence of SEQ ID No. 1 is as Follows:

Phe Asp Thr Gly Ser Ser Asp Leu Leu Val Pro Ser Ile Asn

### The Amino Acid Sequence of SEQ ID No. 2 is as Follows:

Ser Phe Leu Lys Glu His Ala Tyr Arg Leu Ser Gln Ile Ser Phe Arg Ala Ser Asn Leu

Preferably, the polypeptide antigen with the amino acid sequence of SEQ ID No. 1 is selected to prepare a labeled antibody for a sandwich assay detection, and the polypeptide antigen with the amino acid sequence of SEQ ID No. 2 is selected to prepare a coated antibody for the sandwich assay detection.

The present disclosure also provides a monoclonal antibody for detecting bovine and sheep pregnancy-associated glycoprotein prepared by using the co-antigen polypeptides mentioned-above; and the application of the monoclonal antibody in the preparation of a test strip or a detection kit.

The present disclosure also provides a test strip for detecting bovine and sheep PAG; the test strip comprises a sample pad, a binding pad, a coating membrane, and a water absorption pad; wherein the binding pad is coated with a labeled antibody, which can bind specifically to the polypeptide antigen with amino acid sequence SEQ ID No. 1 and the coated membrane is coated with a coated antibody, which can bind specifically to the polypeptide antigen with amino acid sequence SEQ ID No. 2.

The beneficial effects of the present disclosure are: antibodies prepared by using the polypeptide antigen provided by the present disclosure can be used for simultaneously detecting early pregnancy of bovine and sheep; the test strip of the present disclosure fills the blank in the prior art, and can combine two commercial tests in a single test strip, and improves the convenience of customer use; on the other hand, the production cost of manufacturers is also reduced.

### BRIEF DESCRIPTION OF THE DRAWINGS

FIG. 1 is a schematic diagram of the structure of colloidal gold test strip.

FIG. 2 is a Four-parameter fitting curve diagram of labeled antibody ELISA detection.

FIG. 3 is a Four-parameter fitting curve diagram of coated antibody ELISA detection.

### DETAILED DESCRIPTION

The present disclosure is described in detail in combination with the embodiments and attached drawings. The following embodiments are implemented on the premise of the technical scheme, and the detailed implementations and specific operation processes are given. However, the scope of protection of the present disclosure is not limited to the following embodiments.

In the following embodiments, unless otherwise specified, all methods are conventional methods; the reagents and materials described, unless otherwise specified, can be obtained from commercial sources.

### Embodiment 1

Preparation of the monoclonal antibodies against bovine and sheep pregnancy-associated glycoproteins.
1. Coupling of the polypeptide with the carrier protein: the inventor of the present application redesigns the antigen polypeptide sequence by studying the existing competitive product in combination with computer simulation. Polypeptide 1 (SEQ ID No. 1) and Polypeptide 2 (SEQ ID No. 2) are obtained by artificial synthesis, and the Polypeptides and the carrier protein are coupled to prepare antigen polypeptide 1-KLH (keyhole limpet hemocyanin), polypeptide 1-OVA (ovalbumin), polypeptide 2-BSA (bovine serum albumin), polypeptide 2-OVA (SMCC activated carrier protein, Sulfhydryl on Cysteine of Amino and Polypeptide of Protein Linked by SMCC);
2. Preparation of the monoclonal antibody: mixing the polypeptide 1-KLH, the polypeptide 2-BSA and adjuvant; immunizing BALB/c mice for 4 times; respectively coating the polypeptide 1-OVA and the polypeptide 2-OVA to detect the serum titer; if the titer is low, continuing to strengthen immunity 1-2 times, and if the serum titer reaches 1:128000, the immune is qualified; preparing for cell fusion. Selecting a mouse with the highest titer; the spleen of the mouse is stripped by the sterile operating table; preparing spleen cells and myeloma cells; performing electrofusion; the fused cells are resuspended in HAT screening culture medium, and then laid on 10 96-well plates, cultured at 37° C. and 5% CO₂; employing the HAT screening medium for semi-liquid exchange on the third day; employing the HAT screening medium to perform full exchange on the fifth day; and sucking 100 μL of supernatant in each hole for detection on the eighth day; the detection steps are as follows:
(1) coating a 96-well ELISA plate with the polypeptide 1-OVA and the polypeptide 2-OVA, adding the 100 μL fused supernatant into each coated plate, and incubating the 96-well ELISA plate at 37° C. for 1 h; (2) throwing away the supernatant, washing the plate with a washing liquid for three times, and drying; (3) adding a secondary antibody, and incubating the 96-well ELISA plate at 37° C. for 0.5 h; (4) throwing away the supernatant, washing the plate with a washing liquid for 3 times, and drying; (5) adding a color developing solution, 100 μL/well, and incubating the 96-well ELISA plate at 37° C. in dark for 10 min; (6) adding a 50 μL/well of terminating solution 2M HCl; (7) Placing the 96-well ELISA plate in a microplate reader and reading at 450-630 nm.

Selecting a plurality of positive holes according to the microplate reader readings, discarding the liquid in the corresponding numbered 96-well plate holes, re-supplementing 200 μL/well of the hole HAT screening medium, performing re-inspection at 37° C. and 5% CO₂; sucking the cell supernatant on the next day for re-inspection, and during re-inspection, using a non-specific polypeptide-BSA coated plate as a negative screening; filtering out clones that recognize the linker site between the peptide and the carrier, retaining only clones positive for the target peptide-BSA; transferring the corresponding 96-well cells to 24-well enlarged culture, and cryopreserving the cells when the cells are cultured to 80% of the bottom area of the hole; Selecting the cells that need to be subcloned, resuscitating them; performing subcloning by means of a limited dilution method with an HT culture medium; placing a subcloning plate at 37° C. and a 5% CO₂ incubator for about 8 days, taking a cell supernatant for detection, and picking a positive hole to continue subcloning until a monoclonal cell line is obtained; expanding and culturing the monoclonal cell line; preparing a monoclonal antibody by means of suspension culture or ascites; purifying the monoclonal antibody by PROG to obtain a monoclonal antibody, and performing concentration detection on the monoclonal antibody by using a UV method.

### Embodiment 2

### Preparation of Test Strip for Detecting Bovine and Sheep PAG

1. Preparation of colloidal gold solution: taking a clean round-bottom flask; adding double distilled water; placing the clean round-bottom flask on a magnetic heating stirrer, heating and stirring it until boiling; adding 1% chloroauric acid solution to a final concentration of 1/10,000, and continuing to heat until boiling; then adding 1% trisodium citrate solution at a ratio of 1:1.6, continuing to heat and boil for about 10 minutes, until the solution turns a translucent red color. After cooling to room temperature (15° C.-25° C.), diluting above mixed solution to the original planned volume with double distilled water (compensate for evaporation losses), thereby obtaining a colloidal gold solution.

2. Preparation of colloidal gold conjugate pad: placing colloidal gold solution on a magnetic stirrer; adjusting pH to about 8.2 with 0.2M K₂CO₃; adding 5 μg/ml PAG monoclonal antibody 1 (labeled antibody prepared using the Polypeptide 1) under stirring, stirring at room temperature for 30 minutes; adding 10% bovine serum albumin (BSA) solution to a final concentration of 1%; stirring at room temperature for 30 minutes; centrifuging the solution at 4° C. and 10000r/min for 30 minutes; discarding the supernatant, resuspending the precipitate with 1% colloidal gold solution, and mixing well. Using a UV spectrophotometer to test the OD value of the gold-labeled conjugate, using a gold spray membrane instrument to spray the gold-labeled conjugate diluted to an OD value of 1.0 on a 10 mm*30 cm glass cellulose membrane at a spray volume of 2 μl/cm; placing the glass cellulose membrane in a blast drying oven at 37° C. and a humidity of 10%-30% for 24 hours, and setting aside.

3. Preparation of coating membrane: diluting goat anti-mouse IgG monoclonal antibody and PAG monoclonal antibody 2 (coating antibody prepared using the Polypeptide 2) to final concentrations of 0.3 mg/ml and 0.5 mg/ml respectively with coating solution and spraying them on the corresponding positions of nitrocellulose membrane using a gold spray membrane streaking instrument at an amount of 1 μL/cm to prepare C line and T line; drying the membrane in a forced air-drying oven at 37° C. and 10%-30% humidity for 24 hours; setting aside.

4. Preparation of the sample pad: Preparing the sample pad treatment solution, evenly applying 50 ml of the sample pad treatment solution on a 25 cm*30 cm glass cellulose membrane; placing the glass cellulose membrane in a drying room at room temperature with a humidity of 10%-30% for 15 hours-18 hours and setting aside.

5. Large board assembly: cutting the processed sample pad into 3.0 cm*30 cm specifications; the nitrocellulose membrane specifications are 2.5 cm*30 cm; the absorbent pad specifications are 1.7 cm*30 cm; the PVC base plate specifications are 6.0 cm*30 cm; assembling a absorbent paper, a NC membrane, a binding pad, and a sample pad on the PVC base plate in order.

6. Test strip assembly: cutting the assembled large board into 3.00 mm width with a strip cutter and put it into a plastic card shell. The assembled card shell and desiccant are packed into an aluminum foil bag and sealed.

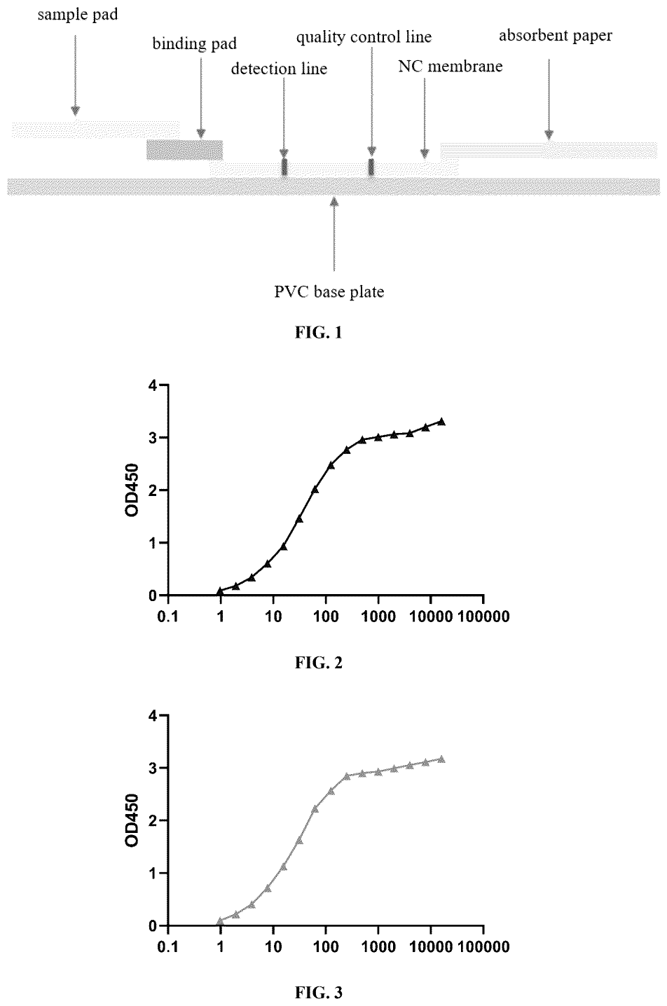

7. Composition of colloidal gold test strip: this test strip consists of a strip body and a plastic card shell, and the plastic card shell consists of an upper shell and a lower shell; a strip body of the test strip composition (see FIG. 1) includes a PVC base plate, a nitrocellulose membrane, a water-absorbing pad, a colloidal gold binding pad, and a sample pad. The colloidal gold binding pad is coated with a PAG monoclonal antibody-colloidal gold marker, and the nitrocellulose membrane is coated with a quality control line C line composed of a sheep anti-mouse IgG monoclonal antibody and a detection line T line composed of a PAG monoclonal antibody.

### EXPERIMENTAL EXAMPLE

### 1. Antigen-Antibody Affinity Data

According to the saturation concentration method, when using ELISA for affinity determination, an obvious four-parameter S-shaped curve is required, that is, a signal curve that changes gradually with the antibody concentration is required. There are obvious upper and lower plateaus at low and high antibody concentrations. The antibody concentration (EC50) value corresponding to half of the upper plateau signal value is the affinity KD value between the antigen and antibody. The main test steps are as follows:
(1) Preparation of ELISA plate: dissolving polypeptide 1-OVA antigen and polypeptide 2-OVA antigen at room temperature, then mixing well and diluting to 1 μg/ml with coating solution; adding 100 μl/well to a 96-well ELISA plate respectively, and incubating the 96-well ELISA plate at 2-8° C. overnight;
(2) Washing the plate 3 times with washing solution, pat drying; adding 300 μL/well blocking solution, blocking for 1 hour at room temperature; washing the plate 3 times with washing solution, pat drying for later use;
(3) Diluting the antibody 2-fold starting from 16000 ng/ml and performing 15 dilutions; adding the blank dilution solution to the last well as a blank control, 100 μL/well; incubating the plate at 37° C. for 1 h;
(4) Washing the plate 3 times with washing solution, pat drying for later use;
(5) Adding secondary antibody, 100 μL/well; incubating the plate at 37° C. for 0.5 h;
(6) Washing the plate 3 times with washing solution, pat drying for later use;
(7) Adding TMB colorimetric solution, 100 μL/well; incubating the plate at 37° C. for 10 min;
(8) Adding terminating solution 2M HCl, 50 μL/well;
(9) Reading at OD450-OD630.

The test results are shown in Table 1, FIG. 2 and FIG. 3.

TABLE 1
ELISA results of labeled antibody and coated antibody:

| Antibody concentration | OD450(labeled | OD450(coated |
|---|---|---|
| (ng/ml) | antibody) | antibody) |
| 16000 | 3.33 | 3.20 |
| 8000 | 3.22 | 3.14 |
| 4000 | 3.10 | 3.09 |
| 2000 | 3.08 | 3.02 |
| 1000 | 3.03 | 2.96 |
| 500 | 2.98 | 2.93 |
| 250 | 2.79 | 2.88 |
| 125 | 2.50 | 2.60 |
| 62.5 | 2.02 | 2.26 |
| 31.25 | 1.49 | 1.66 |
| 15.625 | 0.95 | 1.16 |
| 7.8125 | 0.62 | 0.75 |
| 3.90625 | 0.36 | 0.44 |
| 1.953125 | 0.20 | 0.25 |
| 0.9765625 | 0.11 | 0.12 |
| 0 | 0.02 | 0.03 |

Origin8.0 software can be used to fit the four-parameter fitting curves of the two antibody ELISA tests, as shown in FIGS. 2 and 3. The curves in FIGS. 2 and 3 are both standard S-shaped curves. The fitting results show that the affinity KD values of the labeled antibody and the coated antibody are 34.2 ng/ml and 28.5 ng/ml, respectively. The two antibodies have good affinities for the corresponding polypeptide antigens. 2. Performance Verification Data

### 2.1 Sample Coincidence Rate

### (1) Detection Methods

Sample processing method: the test samples can be serum, plasma, or whole blood. For serum or plasma, please test within 4 hours after separation. If it cannot be tested within 4 hours, please store it at 2° C.-8° C. within 5 days; whole blood samples are collected according to conventional methods and should be used as soon as possible after collection. EDTA or sodium heparin can be used to anticoagulate whole blood;

Test strip preparation: restoring the test strip (prepared in Embodiment 2) to room temperature (15° C.˜30° C.) before use. The test should be performed at room temperature. Opening the packaging bag and placing the test strip on a horizontal, dry surface; using it as soon as possible within 1 hour after opening to prevent the test strip from getting damp.

Sample testing: restoring the sample to room temperature, mixing the serum and plasma samples, and then adding 4 drops (about 100 μL) to the sample well with a dropper; adding 4 drops of whole blood sample with a dropper, then adding a drop of whole blood buffer to the sample well, waiting for 15-20 minutes to observe the results, and the test results will be invalid after more than 30 minutes.

Result determination: positive (+): two purple-red bands appear; one is in the test area (T) and the other is in the quality control area (C), which is judged as positive. Negative (−): only one purple-red band appears in the quality control area (C), and no purple-red band appears in the test area (T), which is judged as negative. Invalid: No purple-red band appears in the quality control area (C), indicating an incorrect operation process or the reagent has deteriorated or damaged.

(2) Sample coincidence rate: 200 blood samples of bovine and 200 blood samples of sheep were collected from the ranch 28 days after mating. The colloidal gold test strip (prepared in Embodiment 2), the bovine early pregnancy detection test strip of Beijing Beite Shuang Company, and the sheep early pregnancy rapid detection test strip of Maikuodusi Biological Company were used to test the obtained serum, plasma, and whole blood samples; the concordance rate of the colloidal gold test strip (prepared in Embodiment 2) and test strips of the competitor was calculated.

Table 2 shows the calculation method of the concordance rate between two detection methods (specifically, colloidal gold test strip or fluorescent test strips and commercial colloidal gold test strips).

TABLE 2
Calculation of the concordance rate

| test strip (prepared in | test strips of the competitor | | |
|---|---|---|---|
| Embodiment 2) | Positive | Negative | Total |
| Positive | a | b | a + b |
| Negative | c | d | c + d |
| Total | a + c | b + d | a + b + c + d |

| | |
|---|---|
| Positive concordance rate | a/a + c |
| Negative concordance rate | d/b + d |
| Total concordance rate | a + d/a + b + c + d |

Among them, “a” indicates the number of samples whose test results of both the test strip (prepared in Embodiment 2) and the test strips of the competitor (commercial colloidal gold test strips) are positive; “b” indicates the number of samples whose test results of the r the test strip (prepared in Embodiment 2) are negative and the test results of the test strips of the competitor are positive; “c” indicates the number of samples whose test results of the test strip (prepared in Embodiment 2) are positive and the test results of the test strips of the competitor are negative; “d” indicates the number of samples whose test results of both the test strip (prepared in Embodiment 2) and the test strips of the competitor are negative.

Table 3 lists the conformity rates between the colloidal gold test strip (prepared in Embodiment 2) and the commercial colloidal gold test strip for early pregnancy in bovine. It can be seen from Table 3 that the colloidal gold test strip, compared with the commercial colloidal gold test strip, has a positive conformity rate of up to 98%, a negative conformity rate of up to 96%, and an overall sample conformity rate of up to 97.5%.

TABLE 3
The conformity rate between the colloidal gold test strip
(prepared in Embodiment 2) and the commercial colloidal
gold test strip for early pregnancy in bovine.

| | commercial colloidal gold test strip | | |
|---|---|---|---|
| colloidal gold test strip | for early pregnancy in bovine | | |
| (Embodiment 2) | Positive | Negative | Total |
| Positive | 145 | 2 | 147 |
| Negative | 3 | 50 | 53 |
| Total | 148 | 52 | 200 |

| | |
|---|---|
| Positive concordance rate | 98% |
| Negative concordance rate | 96% |
| Total concordance rate | 97.5% |

Table 4 lists the conformity rates between the colloidal gold test strip (prepared in Embodiment 2) and the commercial colloidal gold detection test strip for early pregnancy in sheep. It can be seen from Table 4 that the colloidal gold test strip (prepared in Embodiment 2), compared with the commercial colloidal gold test strip, has a positive conformity rate of up to 98.5%, a negative conformity rate of up to 95.5%, and an overall sample conformity rate of up to 97.5%.

TABLE 4
The conformity rate between the colloidal gold test strip
(prepared in Embodiment 2) and the commercial colloidal
gold test strip for early pregnancy in sheep.

| | the commercial colloidal gold | | |
|---|---|---|---|
| colloidal gold test strip | test strip for early pregnancy in sheep | | |
| (Embodiment 2) | Positive | Negative | Total |
| Positive | 132 | 3 | 135 |
| Negative | 2 | 63 | 65 |
| Total | 134 | 66 | 200 |

| | |
|---|---|
| Positive concordance rate | 98.5% |
| Negative concordance rate | 95.5% |
| Total concordance rate | 97.5% |

What is claimed is:

1. A polypeptide antigen, which can bind to pregnancy-associated glycoprotein in bovine and sheep, comprising two co-antigen polypeptides, one of the co-antigen polypeptides comprises the amino acid sequence shown in SEQ ID NO. 1, the other comprises the amino acid sequence shown in SEQ ID NO. 2.


SEQUENCE LISTING (2 sequences)

SEQ ID NO: 1 - protein, 14 aa, synthetic construct
FDTGSSDLLVPSIN

SEQ ID NO: 2 - protein, 20 aa, synthetic construct
SFLKEHAYRLSQISFRASNL